(19) United States
(12) Patent Application Publication
(10) Pub. No.: US 2011/0207208 A1
(43) Pub. Date: Aug. 25, 2011

(54) Compostation Apparatus

(76) Inventor: Moshe Shalit, Ayelet Ha Shahar (IL)

(21) Appl. No.: 12/709,546

(22) Filed: Feb. 22, 2010

(51) Int. Cl. C12M 1/02

(52) U.S. Cl. 435/290.2

(57) ABSTRACT

A compostation apparatus comprising a construction enclosing piles of waste delivered by a conveyor, and a stirring device having vertical blades on a horizontal cable arranged to pass through the waste and aerate and vibrate it. The cable is spun between an electromagnet and a spring, and the electromagnet is arrange to pull and abruptly release the cable against the spring such as to vibrate the blades, causing additional breaking down of waste clumps.

### BACKGROUND

1. TECHNICAL FIELD

The present invention relates to the field of compostation and anaerobic digestion, and more particularly, to a stirring and inoculation device.

2. DISCUSSION OF RELATED ART

Prior to setting forth the background of the related art, it may be helpful to set forth definitions of certain terms that will be used hereinafter.

### BRIEF SUMMARY

Embodiments of the present invention provide an apparatus comprising: a construction arranged to enclose at least one pile of waste, the construction having a length axis; a horizontal axis; and a vertical axis, and further an entrance and an exit on opposite sides along the length axis; a conveyor arranged to transport piles sequentially into the construction from the entrance to the exit along the length axis at a specified rate; at least one stirring device arranged to pass through the piles of waste along the length axis and along the vertical axis and loosen the piles such as to enhance composting or anaerobic digestion of the waste, each stirring device comprising: two shafts connected to a motor, the shafts arranged to be moved by the motor along the length axis of the construction, one shaft on each side of the construction; a covered cable movably connected between the shafts such as to move with the shafts along the length axis, and to be movable along the shafts along the vertical axis, the covered cable connected to one of the shafts by a spring and to the other shaft through an electromagnet; a plurality of blades perpendicularly connected to the covered cable and having edges that are oriented along the length axis, such as to cut through the waste as the shafts and the covered cable move along the length axis, wherein the electromagnet is arranged to pull and abruptly release, at specified periods, the covered cable against the spring, such as to move the blades in a specified motion pattern along the horizontal axis and agitate thereby the waste in a surroundings of the blades.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

The present invention will be more readily understood from the detailed description of embodiments thereof made in conjunction with the accompanying drawings of which:

FIG. 1 is a perspective longitudinal section illustration of a compostation apparatus according to some embodiments of the invention;

FIG. 2 is a perspective transversal section illustration of the compostation apparatus according to some embodiments of the invention;

FIG. 3 is a combined perspective longitudinal and transversal section illustration of the compostation apparatus according to some embodiments of the invention;

FIG. 4 is a side view of a longitudinal section illustration of the compostation apparatus according to some embodiments of the invention;

FIG. 5 is a front view of a transversal section illustration of the compostation apparatus according to some embodiments of the invention;

FIG. 6 is a schematic side view of the compostation apparatus according to some embodiments of the invention; and

FIG. 7 is a schematic illustration of an agitator, according to some embodiments of the invention.

### DETAILED DESCRIPTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

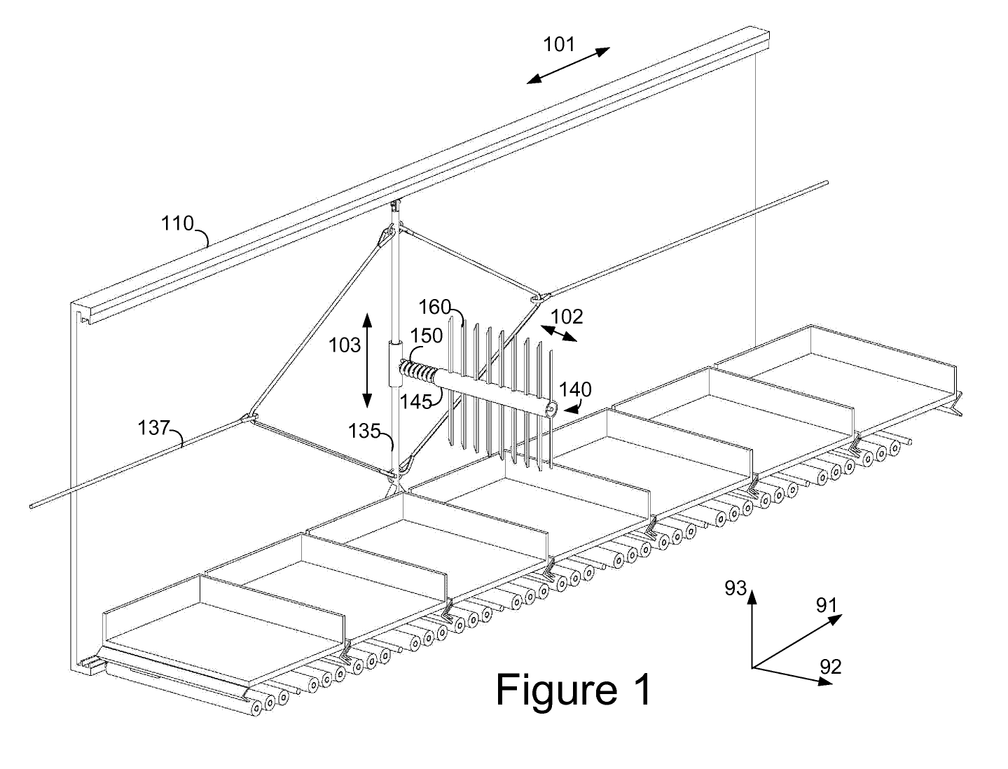

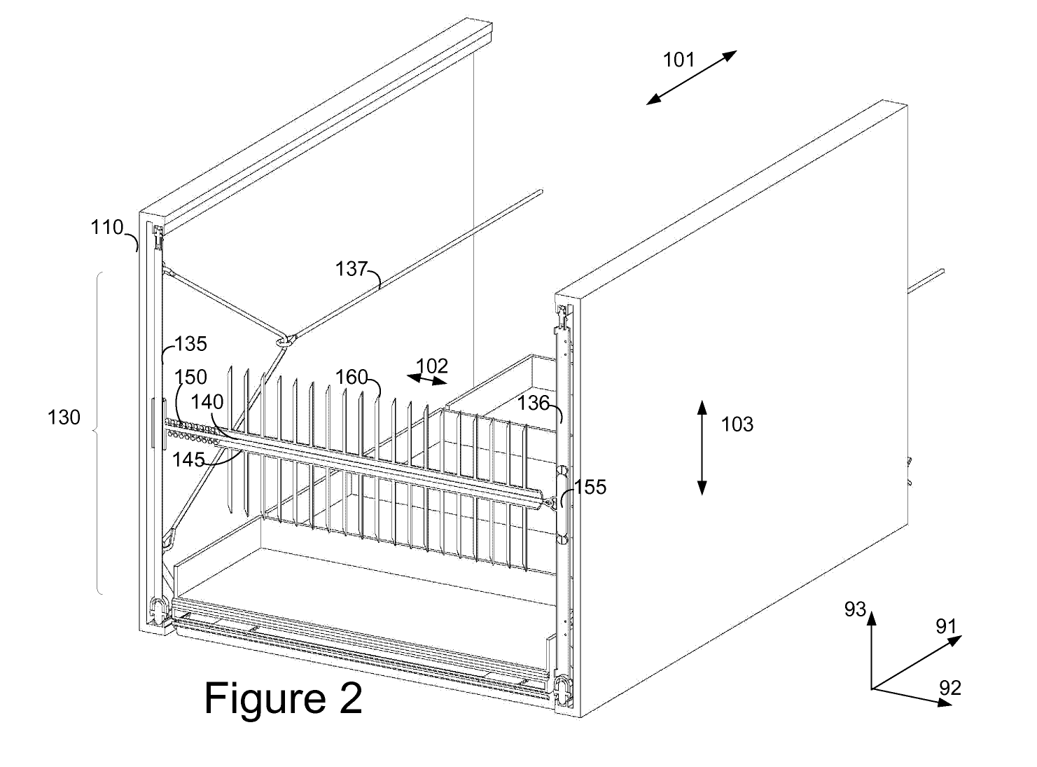

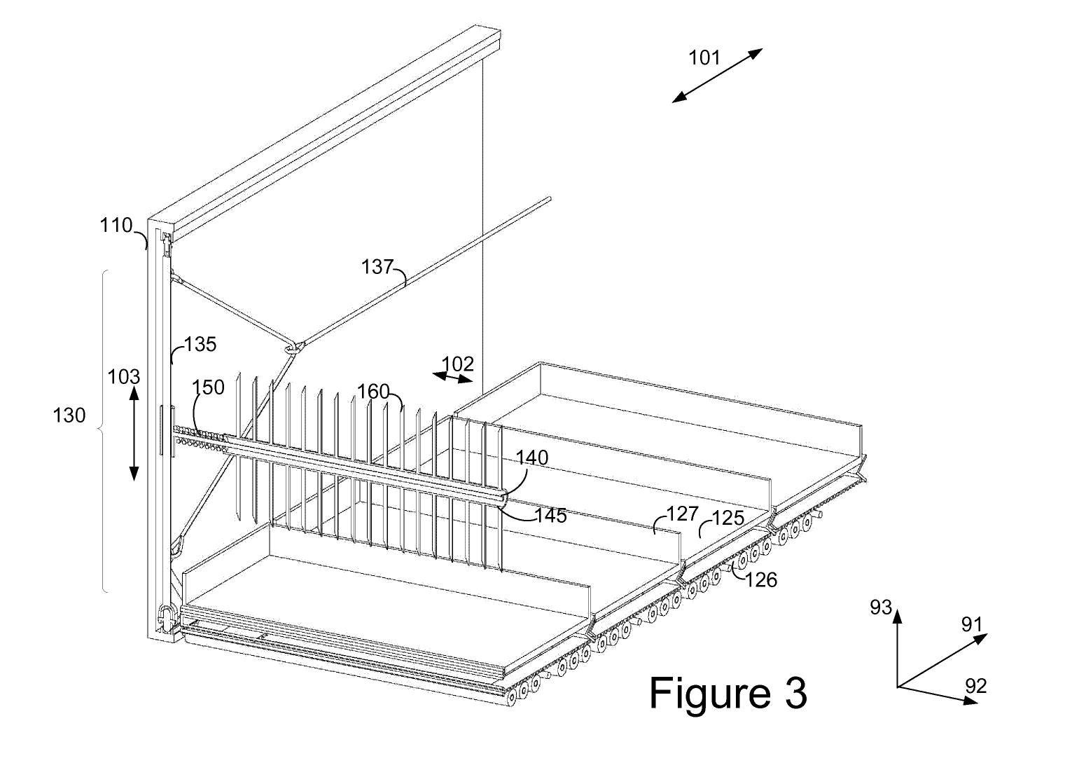

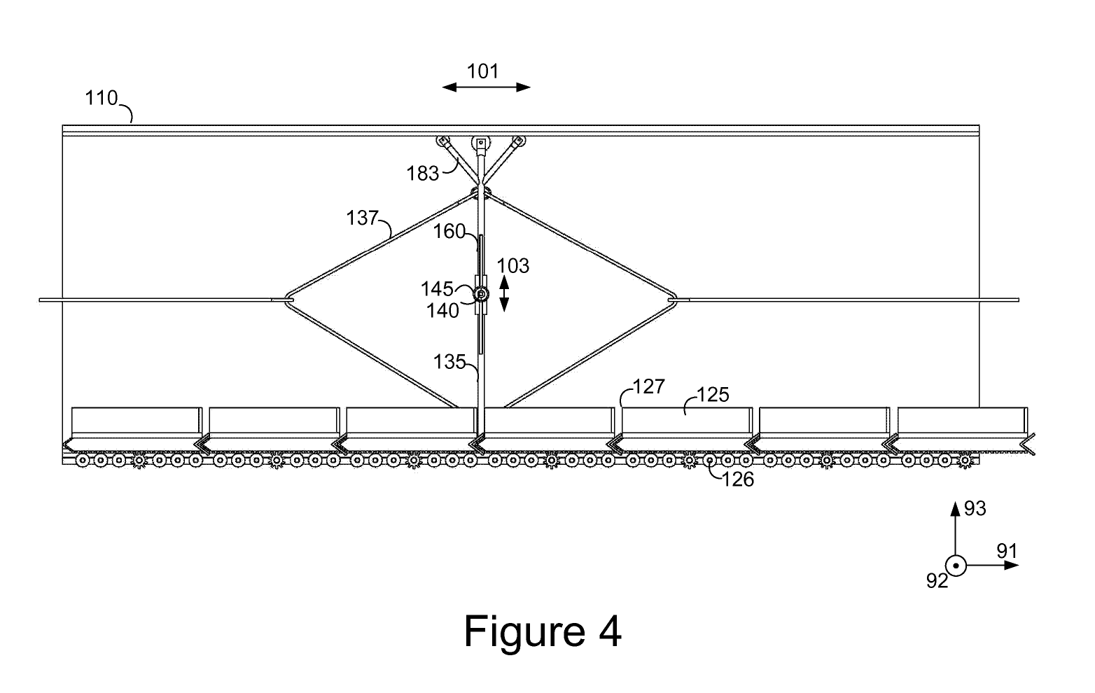

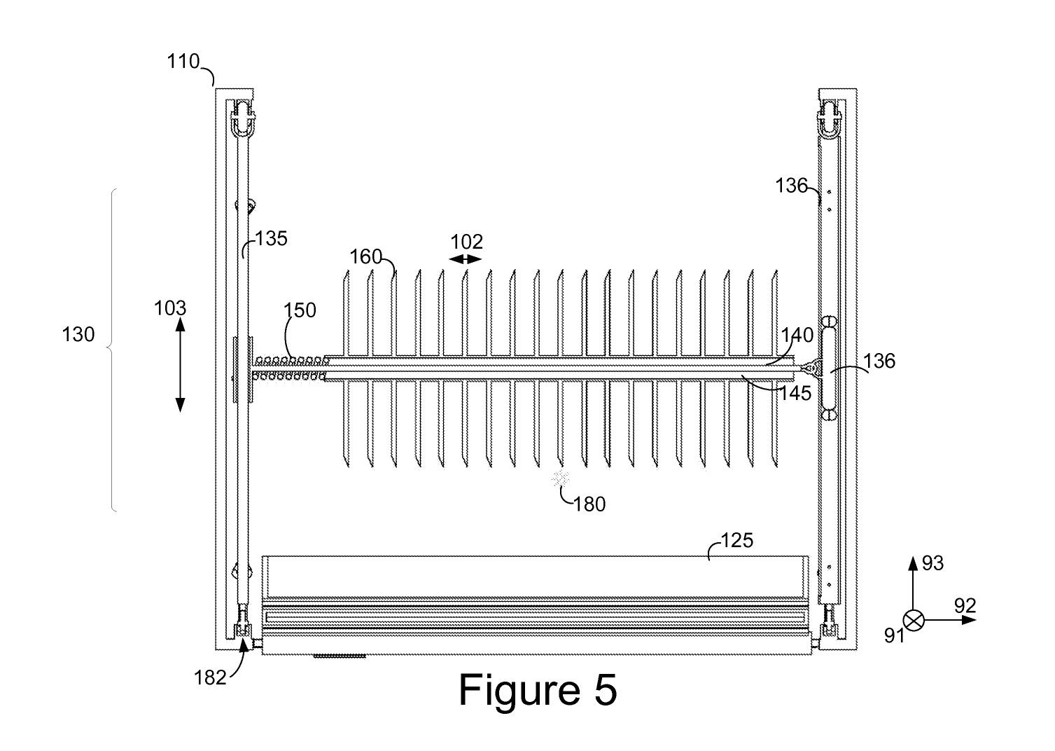

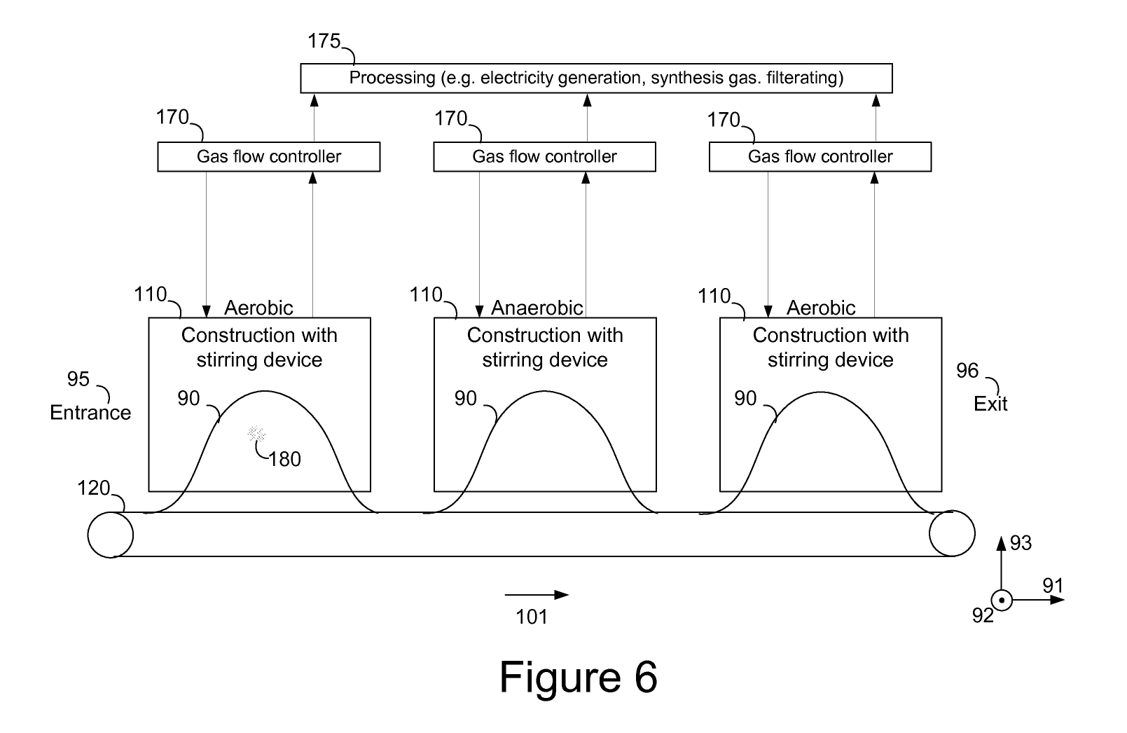

FIGS. 1-6 are high level schematic illustrations of a compostation apparatus 100 according to some embodiments of the invention. FIG. 1 is a perspective longitudinal section, FIG. 2 is a perspective transversal section, FIG. 3 is a combined perspective longitudinal and transversal section, FIG. 4 is a side view of a longitudinal section, FIG. 5 is a front view of a transversal section, and FIG. 6 is a schematic side view of apparatus 100.

Compostation apparatus 100 comprises a construction 110 enclosing piles 90 of waste delivered by a conveyor 120, and a stirring device 130 having vertical blades 160 on a horizontal cable 140 arranged to pass through the waste and aerate it. Cable 140 is spun between an electromagnet 155 and a spring 150, and electromagnet 155 is arrange to pull and abruptly release cable 140 against spring 150 such as to vibrate blades 160, causing additional breaking down of waste clumps.

Apparatus 100 comprises construction 110 arranged to enclose at least one pile 90 of waste. Construction 110 has a length axis 91; a horizontal axis 92; and a vertical axis 93, and further an entrance 95 and an exit 96 on opposite sides along length axis 91.

Apparatus 100 comprises conveyor 120 arranged to transport piles 90 sequentially into construction 110 from entrance 95 to exit 96 along length axis 91 at a specified rate. The specified rate may be one pile daily.

Apparatus 100 comprises at least one stirring device 130 arranged to pass through piles 90 of waste along length axis 91 (arrow 101) and along vertical axis 93 and loosen piles 90 such as to enhance composting of the waste. Stirring device 130 may enhance either aerobic or anaerobic digestion of the waste.

Each stirring device 130 comprises two shafts 135, 136 connected to a motor (not shown) via e.g. cables 137. Shafts 135, 136 are arranged to be moved by the motor along length axis 91 of construction 110 (arrow 101), one shaft 135, 136 on each side of construction 110.

Stirring device 130 comprises covered cable 140 movably connected between shafts 135, 136 such as to move with shafts 135, 136 along length axis 91 (arrow 101), and to be movable along shafts 135, 136 along vertical axis 93 (arrow 103).

Covered cable 140 may be a metal cable covered by a plastic covering 145.

Covered cable 140 is connected to one of the shafts 135 by spring 150 and to other shaft 136 through electromagnet 155.

Stirring device 130 comprises a plurality of blades 160 perpendicularly connected to covered cable 140 and having edges 161 that are oriented along length axis 91, such as to cut through piles 90 as shafts 135, 136 and covered cable 140 move along length axis 91 (arrow 101).

Electromagnet 155 is arranged to pull and abruptly release, at specified periods, covered cable 140 against spring 150, such as to move blades 160 in a specified motion pattern along horizontal axis 92 (arrow 102) and agitate thereby the waste in a surroundings 163 of blades 160.

Electromagnet 155 and spring 150 are arranged to move along shaft 135, 136 together with covered cables 140 (arrow 103).

Construction 110 may be operatively sealed and further comprise controllers 170 for regulating gas inflow and outflow. Aeration conditions within construction 110 may be thus controlled such as to provide an appropriate environment for fermentation processes. For example, construction 110 may be controlled to support aerobic or anaerobic compostation.

Outflowing gas may be collected and processed 175, e.g., used for electricity generation, for generation of synthesis gas, or filtered.

Construction 110 may comprise several chambers in line, each with its own stirring device 130 and a common conveyor 120 arranged to transport piles 90 from one chamber to the other along the line. The chambers may differ in their aeration conditions (e.g., some having aerobic conditions, other having anaerobic conditions, optionally with adequate fermentation in respect to the aeration conditions).

Sealing of construction 110 may be carried out by sealing the entrance and the exit of each chamber during operation, and opening them only to admit and release piles 90.

Conveyor 120 may comprise a plurality of interconnected platforms 125, each platform 125 in a size of pile 90, and with an integer number of platforms 125 fitting into construction 110, such as to allow sealing construction 110. Groups of chambers within construction 110 may be similarly sealed. Platforms 125 may be mounted on cylindrical rollers 126.

Operating apparatus 100 comprises entering piles 90 stepwise into construction 110, such that construction 110 is kept sealed except for brief periods of entering piles 90. Construction 110 may comprise a door opening only to accept pile 90, and the door may be sealed against an edge 127 of platform 125.

Construction 110 may comprise a fermentation agent (not shown) arranged to fermentate piles 90 during their passage through construction 110.

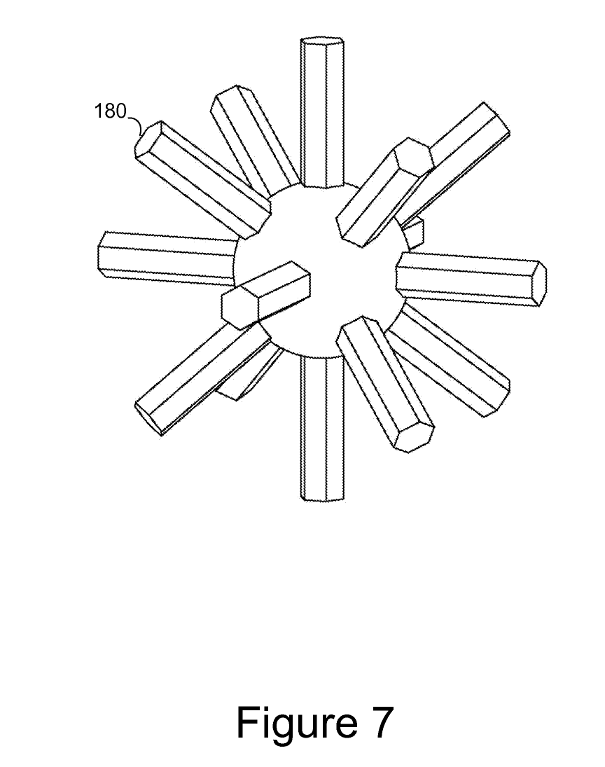

Apparatus 100 may further comprise a plurality of agitators 180 (FIGS. 5, 6 and 7). Agitators 180 may be small balls with multiple spines that may be impregnated with cultures of microorganisms that are selected according to the aims of compostation (e.g., after the type of aeration, the required gas products, or the types of waste). The cultures of microorganisms may be selected such as to enhance either aerobic or anaerobic digestion of the waste, or a composition thereof in apparatus 100 with groups of compartments with differing aeration conditions.

Agitators 180 may be designed such as to be moved by blades 160 and further break down waste clumps in a smaller scale that that affected by blades 160. For example, agitators 180 may have an overall smaller diameter that the distance between adjacent blades 160.

Agitators 180 may be separated from the waste at exit 96, processed, and returned into new pile 90 at entrance 95.

Blades 160 and covered cable 140 are designed such as to minimize friction as stirring device 130 moves through the waste. Waste may comprise up to 40% solids.

Shafts 135, 136 may be mounted on tracks and may have e.g., pinions 182 on their bottom moving on bottom racks (FIG. 5), and guiding wheels 183 (FIG. 4) on their top moving in upper tracks.

In the above description, an embodiment is an example or implementation of the inventions. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not to be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention may be implemented in the testing or practice with methods and materials equivalent or similar to those described herein.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

What is claimed is:

1. An apparatus comprising:
a construction arranged to enclose at least one pile of waste, the construction having a length axis; a horizontal axis; and a vertical axis, and further an entrance and an exit on opposite sides along the length axis;
a conveyor arranged to transport piles sequentially into the construction from the entrance to the exit along the length axis at a specified rate;
at least one stirring device arranged to pass through the piles of waste along the length axis and along the vertical axis and loosen the piles such as to enhance composting of the waste, each stirring device comprising:
two shafts connected to a motor, the shafts arranged to be moved by the motor along the length axis of the construction, one shaft on each side of the construction;
a covered cable movably connected between the shafts such as to move with the shafts along the length axis, and to be movable along the shafts along the vertical axis, the covered cable connected to one of the shafts by a spring and to the other shaft through an electromagnet;
a plurality of blades perpendicularly connected to the covered cable and having edges that are oriented along the length axis, such as to cut through the waste as the shafts and the covered cable move along the length axis,
wherein the electromagnet is arranged to pull and abruptly release, at specified periods, the covered cable against the spring, such as to move the blades in a specified motion pattern along the horizontal axis and agitate thereby the waste in a surroundings of the blades.

2. The apparatus of claim 1, wherein the covered cable comprises a metal cable covered by a plastic covering.

3. The apparatus of claim 1, wherein the construction is operatively sealed and further comprise at least one controller arranged to regulate gas inflow and outflow.

4. The apparatus of claim 3, wherein the at least one controller is arranged to maintain in the construction at least one of: aerobic conditions; and anaerobic conditions.

5. The apparatus of claim 3, further comprising a gas processor arranged to process the outflowing gas.

6. The apparatus of claim 5, wherein gas processing comprises at least one of: filtering;
electricity generation; and generation of synthesis gas.

7. The apparatus of claim 1, wherein the construction comprises a plurality of sealed chambers in line, each with one of the at least one stirring devices, and each having specified aeration conditions, wherein the conveyor is arranged to move the piles from chamber to chamber.

8. The apparatus of claim 1, wherein the conveyor comprises a plurality of interconnected platforms, each platform in the size of pile.

9. The apparatus of claim 1, wherein the construction comprises a fermentation agent arranged to fermentate the piles during their passage through the construction.

10. The apparatus of claim 1, further comprising a plurality of agitators inserted into the piles and arranged to be moved by the blades and further break down waste clumps in a smaller scale that that affected by the blades.

11. The apparatus of claim 10, wherein the agitators comprise balls with multiple spines.

12. The apparatus of claim 10, wherein the agitators are further impregnated with cultures of microorganisms.

13. The apparatus of claim 12, wherein the cultures of microorganisms are selected such as to enhance aerobic digestion of the waste.

14. The apparatus of claim 12, wherein the cultures of microorganisms are selected such as to enhance anaerobic digestion of the waste.

15. The apparatus of claim 1, wherein the blades and the covered cable are designed such as to minimize friction as the stirring device moves through the waste.

16. The apparatus of claim 1, wherein the shafts are mounted by pinions on bottom racks, and by guiding wheels on upper tracks.

17. The apparatus of claim 1, wherein the at least one stirring device is arranged to enhance aerobic digestion of the waste.

18. The apparatus of claim 1, wherein the at least one stirring device is arranged to enhance anaerobic digestion of the waste.
